(19) United States
(12) Patent Application Publication
(10) Pub. No.: US 2011/0215215 A1
(43) Pub. Date: Sep. 8, 2011

(54) Securement of Laparoscopic Instruments on the Sterile Surgical Field

(76) Inventor: Scott Laker, Birmingham, MI (US)

(21) Appl. No.: 13/087,325

(22) Filed: Apr. 14, 2011

Related U.S. Application Data
(63) Continuation-in-part of application No. 12/912,687, filed on Oct. 26, 2010.
(60) Provisional application No. 61/255,595, filed on Oct. 28, 2009.

(51) Int. Cl. A61B 19/00; B23P 11/00; F16M 13/00

(52) U.S. Cl. 248/346.5; 29/446

(57) ABSTRACT

Apparatus and associated methods involve holding apparatus that provide non-visual feedback to confirm securement of a surgical tool or instrument at a location in the sterile field. In an illustrative example, a surgical team member may be able to securely load and/or unload a laparoscopic instrument in some embodiments with a single hand by manipulating a proximal handle of the instrument. In some examples, the holding apparatus may provide an audible, visual, or tactile feedback when an instrument is securely loaded or when it is successfully disengaged. In some embodiments, the holding apparatus may store more than one instrument. Accordingly, instruments may be securely stored within a sterile field for convenient access during an operation.

### CROSS-REFERENCE TO RELATED APPLICATIONS

This application is a continuation-in-part (CP) of U.S. Non-Provisional Patent Application entitled “Securement of Laparoscopic Instruments on the Sterile Surgical Field,” Ser. No. 12/912,687, which was filed by Scott Laker on Oct. 26, 2010. This application also claims the benefit of U.S. Provisional Patent Application entitled “Device for Securing Laparoscopic Instruments on the Sterile Field during Laparoscopic Surgery,” Ser. No. 61/255,595, which was filed by Scott Laker on Oct. 28, 2009. The entire contents of each of these applications is incorporated herein by reference.

### TECHNICAL FIELD

Various embodiments relate generally to systems and methods for secure placement of instruments for use in surgery.

### BACKGROUND

During a typical surgery, a patient may be in the supine position (e.g., lying on their back) or prone position (e.g., tying down with the front of their body facing toward a supporting surface). A surgeon may be positioned to the right or the left of the patient, and the assistant may be positioned generally opposite the surgeon. The patient may be covered with a sterile drape from their chest down. The drape may be adhered to the patient's skin with an adhesive. The drape may have an opening over a surgical site (e.g., the patient's abdomen). During a procedure, a variety of instruments may be handled, passed among members of the surgical team, and some instruments may need to be repeatedly retrieved for use and then put aside until needed again. In some examples, instruments not being used may be placed on or near the sterile operative field near the breast bone or lower extremities or nearby surgical tables.

### SUMMARY

Apparatus and associated methods involve holding apparatus that provide non-visual feedback to confirm securement of a surgical tool or instrument at a location in the sterile field. In an illustrative example, a surgical team member may be able to securely toad and/or unload a laparoscopic instrument in some embodiments with a single hand by manipulating a proximal handle of the instrument. In some examples, the holding apparatus may provide an audible, visual, or tactile feedback when an instrument is securely loaded or when it is successfully disengaged. In some embodiments, the holding apparatus may store more than one instrument. Accordingly, instruments may be securely stored within a sterile field for convenient access during an operation.

Certain embodiments of a surgical tool holding apparatus may achieve one or more advantages. For example, various implementations may provide non-visual feedback that confirms to the surgical team member surgeon, assistant, scrub personnel) that the instrument is securely held by the apparatus while the member maintains visual focus on the surgical field or other critical areas. Further, some embodiments may positively locate the instrument in a convenient position for the surgeon to comfortably release the instrument from the apparatus without looking at the instrument. Moreover, some embodiments may provide for single-handed operation to load and/or unload the instrument from the holder. in some examples, the toad and unload operations may require the surgical team member to apply a limited vertical and/or rotational force to a proximal end of the instrument (e.g., handle) both to toad the instrument for storage and/or to release the instrument for use. Some embodiments may have the capacity to simultaneously store several instruments of varying diameter, each of which may be independently stored and retrieved during a surgery. Various examples may present instruments at a secure location for retrieval in a specific order of use at a convenient location on the sterile field. The device may facilitate multiple uses of an instrument by maintaining it in the sterile field in ergonomic proximity to the operating surgeon and/or assistant in a secure manner. The device may substantially reduce the costly consequences of fallen surgical tools that must either be replaced or re-sterilized (e.g., redundancy requirement and increased operative time). Some implementations may reduce potential for injury in handling and/or transfer of sharp instruments among team members. Convenient sterile packaging may include a sterile drape and one or more holder apparatus for convenient preparation of the patient and pre-positioning of surgical instruments. Several embodiments may be formed of low-cost construction that may be partially or completely disposable, although some embodiments may be re-usable by autoclave, for example.

The details of various embodiments are set forth in the accompanying drawings and the description below. Other features and advantages will be apparent from the description and drawings, and from the claims.

### BRIEF DESCRIPTION OF THE DRAWINGS

FIG. 1 is a top view of an exemplary medical instrument holding apparatus.

FIG. 2 is a top view of art exemplary medical instrument holding apparatus with resting pads and a single row of individual retention modules.

FIG. 3 is a side view of an exemplary medical instrument holding apparatus with fulcrums.

FIG. 4 shows a perspective view of an exemplary medical instrument holding apparatus with staggered wall sections.

FIGS. 5A and 5B show perspective views of an exemplary medical instrument holding apparatus with cylindrical wall members.

FIG. 6 is a cross sectional view along the length of a wall section of an exemplary medical instrument holding apparatus showing wall members with an internal rib structure configured to accommodate medical instruments of different shaft diameters and a bilateral cantilever release.

FIG. 7 is a cross sectional view along the length of a wall section of an exemplary medical instrument holding apparatus showing wall members with a circular aperture.

FIG. 8 is a cross sectional view along the length of a wall section of an exemplary medical instrument holding apparatus showing each wall member with a plurality of apertures and a unilateral cantilever release.

FIG. 9 is a cross sectional view along the length of a wall section of an exemplary medical instrument holding apparatus showing each wall member with an aperture in the shape of a gear.

FIG. 10 shows a side view of an exemplary medical instrument holding apparatus that includes a gull winged wall member with an aperture.

FIG. 11 shows a three-dimensional side view of an exemplary gull winged wall member and a fulcrum within the aperture.

FIG. 12 is a side view of an exemplary gull winged wall member with a plurality of apertures.

FIG. 13 is a side view of an exemplary gull winged wall member with ribbed side walls.

FIG. 14 is a side view of an exemplary gull winged wall member with inner surfaces of the wing member's sidewalls ribbed.

FIG. 15 is a side view of an exemplary medical instrument holding apparatus that uses magnetic tubes and stops.

FIGS. 16A and 16B are perspective views of a wall member of a “sliding grip” arrangement.

FIG. 17 is a side view of a wall member of a “spring ball” arrangement.

FIGS. 18A and 18B are side views of a lever operated “spring ball” wall member.

FIGS. 19A and 19B are perspective views of wall members using a “paddle mechanism” to retain medical instruments.

FIG. 20 is a three-dimensional view of an exemplary medical instrument holding apparatus with a parabolic cross section.

FIGS. 21A-21D show perspective views of an exemplary medical instrument holding apparatus.

Like reference symbols in the various drawings indicate like elements.

### DETAILED DESCRIPTION OF ILLUSTRATIVE EMBODIMENTS

In the depicted figures, embodiments of an exemplary medical instrument holding apparatus are configured to securely retain and release medical instrument. Some embodiments are operable to load and/or release an instrument by manipulation using one-hand. Certain embodiments may provide confirmation of the secure retention of the medical instrument by non-visual feedback.

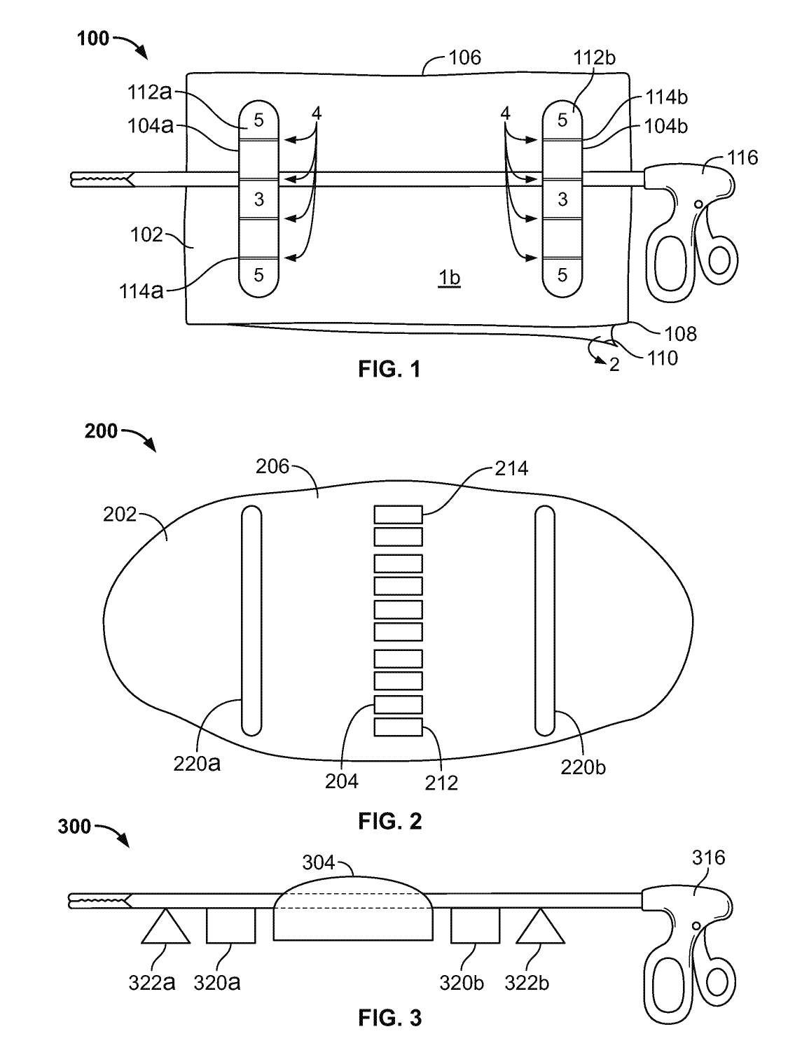

FIG. 1 is a top view of an exemplary medical instrument holding apparatus. As depicted, a medical instrument holding apparatus 100 includes a base member 102 and a pair of wall sections 104a, 104b that extend upwardly from the base member 102 in a substantially perpendicular manner. Each wall section 104a, 104b is positioned longitudinally along opposite sides of the base member 102.

The base member 102 includes a top surface 106 from which the wall sections 104a, 104b extend and a bottom surface 108. The bottom surface 108 may include a sterile, non-reactive adhesive for attaching the medical instrument holding apparatus 100 to the sterile operative field at the discretion of the user. A disposable paper or membrane 110, or liner, may initially cover the adhesive to prevent the adhesive from drying up or sticking to articles before use. The paper or membrane 110 can be peeled off prior to using the medical instrument holding apparatus 100.

Each of the wall sections 104a, 104b may be formed from a plurality of wall members 112a, 112b, respectively, that are spaced apart to form a plurality of apertures or “keyhole” openings 114a, 114b, respectively. Wall members and their associated aperture may also be referred to as a retention module. Each of the apertures 114a of wall section 104a may be laterally aligned with each of the apertures 114b of wall section 104b to retain a medical instrument 116 in two positions, such as the proximal and distal portions of the shaft of the medical instrument 116. The wall members 112a, 112b may be formed from a resilient material that can stretch to accommodate instruments of different diameters and return to substantially its unstretched form to securely enclose instruments.

The wall members 112a, 112b may be generally rectangular members permanently or detachably attached to the top surface 106 of the base member 102 to form apertures 114a, 114b of rectangular geometry. The wall members 112a, 112b at the ends of the wall sections 104a, 104b may have a slight curvature. The top surfaces of the wall members 112a, 112b positioned at either or both ends of the wall sections 104a, 104b may include flattened regions to which counter-tension may be applied by a user to ease securing and releasing the medical instrument without lifting up on the sterile drape, for example. In an illustrative example, the user may place their thumb or other finger on a flattened region while depressing the instrument into an aperture or lift the instrument out of the aperture using one hand without disturbing the surgical drape. The flattened regions may be contoured or textured to enhance the user's grip on the flattened regions.

The base member 102 and wall sections 104a, 104b may be made, in various examples, from inert, sterile, and/or latex free materials, including by way of example and not limitation, polymers such as foam. Each of the base member and wall sections may have the same or different materials. The base member 102 may be a flat rectangular sheet although other arrangements may be contemplated, including elliptical and polygonal. The wall members 112a, 112b may have other geometries to form differently shaped apertures 114a, 114b, such as circular, pyramidal, or polygonal.

In some embodiments, a medical instrument holding apparatus may have additional wall sections to secure a. medical instrument at additional locations, such as the intermediate portion of the medical instrument's shaft. In other embodiments, a medical holding apparatus may have only one wall section, which may be extended in its channel length, to secure and support a medical instrument in a substantially balanced manner.

FIG. 2 is a top view of an exemplary medical instrument holding apparatus with resting pads. As depicted, a medical instrument holding apparatus 200 includes an elliptical base member 202 and wall section 204. The wall section 204 includes a plurality of wall members 212 forming a plurality of apertures 214. The base member 202 includes a top surface 206 to which a pair of resting pad 220a, 220b are attached on opposing sides of the wall section 204 aligned with the apertures 214. The resting pads 220a, 220b may support the medical instrument at positions near the inserted portion of the shaft to secure the instrument against movement, including rotational and translational movement. The resting pads 220a, 220b may be made from materials with friction to resist movement of the inserted medical instrument. In operation, the resting pads may provide a surface to counteract rotation at a distal end of the instrument so that a lifting three at the opposing proximal end of the instrument may lift the instrument out of the channel in the aperture 214 in which the instrument is securely held.

The resting pads 220a, 220b may be placed on both sides of all wall sections. In some embodiments, the resting pads are placed on one side of a wall section. For example, in a medical instrument holding apparatus with two wall sections, a resting pad may be placed on the side of each watt section that is closest to the edge of the base member. A resting pad may be a strip of material aligned with each of the apertures. In other embodiments, multiple resting pads may be used such that each aperture is aligned with at least one resting pad. This may reduce the amount of material used for the resting pads. The separation between the resting pad 220a and an associated wall section 204 may be based on the length of instrument to be secured.

FIG. 3 is a side view of an exemplary medical instrument holding apparatus with fulcrums. A medical instrument holding apparatus 300 includes a wall section 304 retaining a medical instrument 316. The medical instrument holding apparatus 300 includes a pair of resting pads 320a, 320b and a pair of fulcrums 322a, 322b. The pair of resting pads 320a, 320b is positioned on opposing sides of the wall section 304. The resting pad 320a is positioned between the wall section 304 and fulcrum 322a, and the resting pad 322b is positioned between the wall section 304 and fulcrum 322b.

The pair of fulcrums 322a, 322b may ease release of the medical instrument 316 from the medical instrument holding apparatus 300. When a user applies a three to the medical instrument 316 resting upon fulcrums 322a, 322b, that force is multiplied against the weight of the instrument and resistance of the instrument from being secured in the wall section so less effort is required to release medical instrument 316 from the medical instrument holding apparatus 300. In some embodiments, a single fulcrum may be used. For example, a single fulcrum 322b may be positioned between the resting pad 320b and the portion at which force is applied to the medical instrument 316.

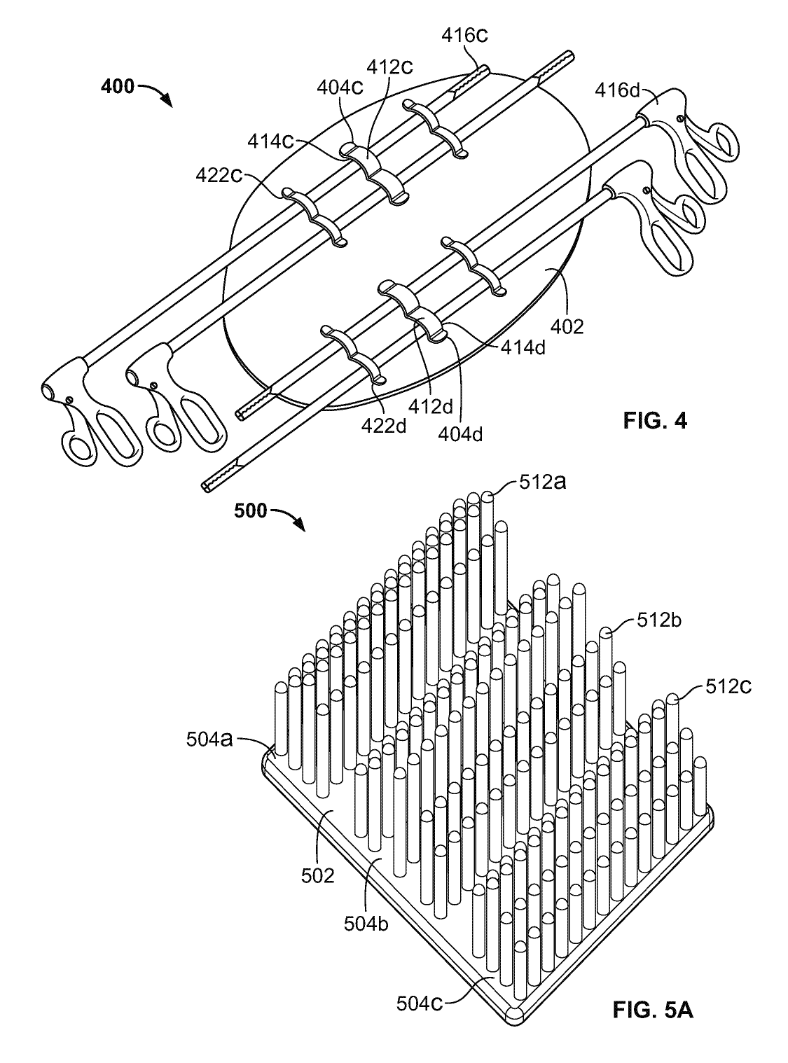

FIG. 4 shows a perspective view of an exemplary medical instrument holding apparatus with staggered wall sections. A medical instrument holding apparatus 400 includes a base member 402 and a pair of wall sections 404c, 404d extending from the base member 402. Each of the wall sections 404c, 404d that may be formed from wall members 412c, 412d, respectively, that are spaced apart to form apertures 414c, 414d, respectively, for receiving a pair of medical instruments 416c, 416d. The medical instruments within a wall section may have different lengths or secured at different positions to prevent overlap of the handles within the wall section. The medical instruments may be secured in one place along the intermediate portion of each medical instrument's shaft. A pair of fulcrums 422c is positioned on opposing sides of wall section 404c, and a pair of fulcrums 422d is positioned on opposing sides of wall section 404d.

The wall section 404c is staggered or offset relative to the wall section 404d on the base member 402 to prevent overlap in the handles of the medical instruments retained in wall section 404c with the handles of the medical instruments retained in wall section 404d. The pair of fulcrums 422c may be correspondingly staggered or offset relative to the pair of fulcrums 422d.

In some embodiments, each of the wall sections may have two spaced apart wall members to form one aperture for receiving one medical instrument. In this example, a plurality of wall sections may be used to accommodate a plurality of medical instruments.

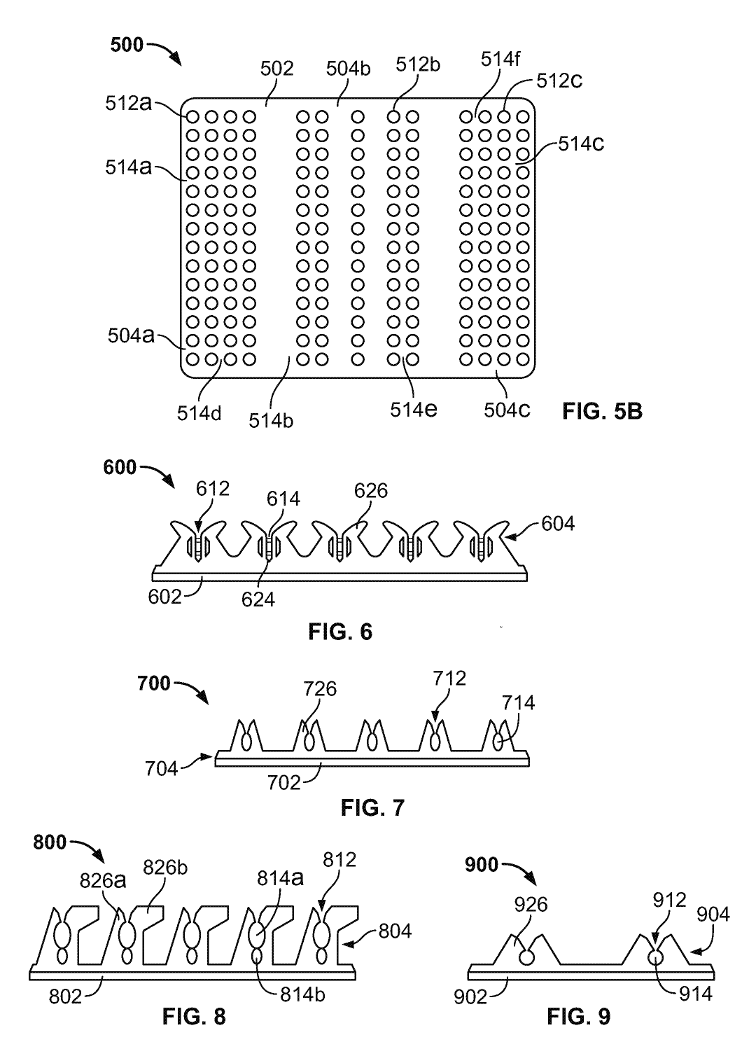

FIGS. 5A and 5B show perspective views of an exemplary medical instrument holding apparatus with cylindrical wall members. FIG. 5A is a three-dimensional perspective view of an exemplary medical instrument holding apparatus. FIG. 5B is a top view of the exemplary medical instrument holding apparatus of FIG. 5A.

A medical instrument holding apparatus 500 includes a base member 502 and wall sections 504a, 504b, 504c that extend upwardly from the base member 502 in a substantially perpendicular manner. The wall sections 504a and 504c are positioned on opposite sides of the base member with wall section 504b positioned between wall sections 504a and 504c.

Each of the wall sections 504a, 504b, 504c includes a plurality of wall members 512a, 512b, 512c, respectively, that are spaced apart to form a set of apertures 514a, 514b, 514c. The apertures in each set 514a, 514b, 514c are laterally aligned with each other to form a channel that can receive the medical instrument (e.g., in the left-to-right orientation in the depicted example). The wall members may be cylindrical projections extending from the base member. The wall members may have the same or different height.

Wall sections 504a, 504c may include a plurality of wall members 512a, 512c arranged in four columns. Wall section 504b may include a plurality of wall members 512b arranged in five columns. In some implementations, medical instruments may be placed in the channels 514d, 514e, 514f between the columns of wall members (e.g., in an orientation orthogonal to the left-to-right orientation). The wall members 512a, 512c in wall sections 504a, 504c may be spaced equidistantly from each other. The outer two columns of wall members 512b in wall section 504b may have substantially the same spacing as the wall members 512a, 512c in wall sections 504a, 504c. The middle column of wall members 512b in wall section 504b may have a wider spacing than its outer two columns. The channel between the middle column await members 512b and the outer two columns may accommodate instruments of larger diameter than other channels between the columns of wall members.

FIGS. 6-9 show perspective views of different retaining features of walls members forming walls sections of different embodiments of medical instrument holding apparatuses.

FIG. 6 is across sectional view along the length of a wall section of an exemplary medical instrument holding apparatus showing wall members with an internal rib structure configured to accommodate medical instruments of different shaft diameters. An exemplary medical instrument holding apparatus 600 includes abase member 602 and wall section 604 extending from the base member 602. The wall section 604 may include a plurality of wall members 612 extending from the base member 602.

Each wall member 612 includes an aperture 614 that is defined by a pair of spaced apart internal ribs 624 to accommodate instruments of differently sized diameters. The ribs 624 may be made from a resilient material that can stretch and return to its unstretched state. A medical instrument with a 10 mm diameter shaft in the aperture 614 may outwardly expand or stretch the ribs 624 more than a medical instrument with a 5 mm diameter shaft. The spacing of the ribs to form the aperture 614 and the use of resilient materials may be optimized for medical instruments with shafts of commercially standard sizes.

The upper portion of the wall member 612 is flanked by a pair of wings 626 upon which counter tension may be applied so a user may engage and release a medical instrument from the aperture 614 by applying a downward force using only one hand. For example, the user may apply a downward force on a wing with or without use of their thumb while using their index finger on the same hand to depress the instrument into the aperture. In some examples, the user may apply a downward force on a wing with their thumb while using their index finger on the same hand to lift the instrument out of the aperture.

FIG. 7 is a cross sectional view along the length of a wall section of an exemplary medical instrument holding apparatus showing wall members with a circular aperture. An exemplary medical instrument holding apparatus 700 includes a base member 702 and wall section 704 extending from the base member 702. The wall section 704 may include a plurality of wall members 712 extending from the base member 702.

Each wall member 712 includes a circular aperture 714 for receiving a medical instrument. The wall members 712 may have the same or differently sized apertures to accommodate instrument shafts of different diameters. The wall members 712 may be formed from a resilient material with stretchability and recoil memory to accommodate instruments of different sizes in their apertures. The aperture may have a smaller diameter than the medical instrument with the smallest diameter that is generally in use, such that the aperture can accommodate this instrument as well as the medical instrument with the largest diameter that is generally in use. The wall member may stretch more when accommodating a 10 mm diameter medical instrument shaft than when accommodating a 5 mm diameter medical instrument shaft.

The upper portion of the wall members 712 may have a pair of inwardly sloped projections 726 upon which a user may apply counter tension. A user may apply a lateral and/or downward force to one of the projections to increase the diameter of aperture and depress a medical instrument into the aperture using the index on the same hand. Conversely, the user may apply a lateral and/or downward force to the projection to increase the diameter of the aperture and lift the medical instrument out of the aperture.

FIG. 8 is a cross sectional view along the length of a wall section of an exemplary medical instrument holding apparatus showing each wall member with a plurality of apertures. An exemplary medical instrument holding apparatus 800 includes a base member 802 and wall section 804 extending from the base member 802. The wall section 804 may include a plurality of wall members 812 extending from the base member 802.

Each wall member 812 has two apertures 814a, 814b. A larger aperture 814a toward the upper portion of the wall member 812 is connected to a smaller aperture 814b toward the lower portion of the wall member 812. The smaller aperture 814b accommodates instruments of smaller diameter (e.g., about 5 mm) shafts than the larger aperture 814c (e.g., about 10 mm).

Each wall member 812 is flanked by projections 826a, 826b at its upper portion. The projections 826a, 826b may be viewed as combining the projections of the embodiments of FIG. 6 and FIG. 7. Projection 826a is similar to the projection 726 in FIG. 7 which is inwardly curved. Projection 826b is similar to the projection 626 in FIG. 6 which has a wing configuration.

A user may apply counter tension against wing projection 826a using their thumb while depressing an instrument of 5 mm shaft into the smaller aperture 814b using another finger on the same hand (e.g. index finger). The instrument may enter the smaller aperture 814b from the larger aperture 814a through a connecting bridge. Conversely, the user may apply counter tension against wing projection 826b using their thumb white lifting the instrument of 5 mm shaft out of the smaller aperture 814b using another finger on the same hand (e.g. index finger). An instrument with a larger diameter shaft, such as 10 mm, may be retained in the larger aperture 814a without any instruments in the smaller aperture 814b. Each wall member 812 may simultaneously retain two instruments of differently sized diameters, for example, a medical instrument with a 5 mm diameter shaft in the smaller aperture 814b and a medical instrument with a 10 mm diameter shaft in the larger aperture 814a.

FIG. 9 is a cross sectional view along the length of a wall section of an exemplary medical instrument holding apparatus showing each wall member with an aperture in the shape of a gear. An exemplary medical instrument holding apparatus 900 includes a base member 902 and wall section 904 extending from the base member 902. The wall section 904 may include a plurality of wall members 912 extending from the base member 902.

Each wall member 912 includes an aperture 914 that is in the shape of a gear. The teeth portions of the aperture 914 can grip a medical instrument more securely within the aperture, particularly instruments with shafts of smaller diameter, but may advantageously deform with less force to release the instrument relative to a solid-core construction. The upper portions of the wall members 912 resemble the upper portions of the wall members 712 in FIG. 7 and may be operated in a substantially similar manner to the medical instrument holding instrument apparatus of FIG. 7.

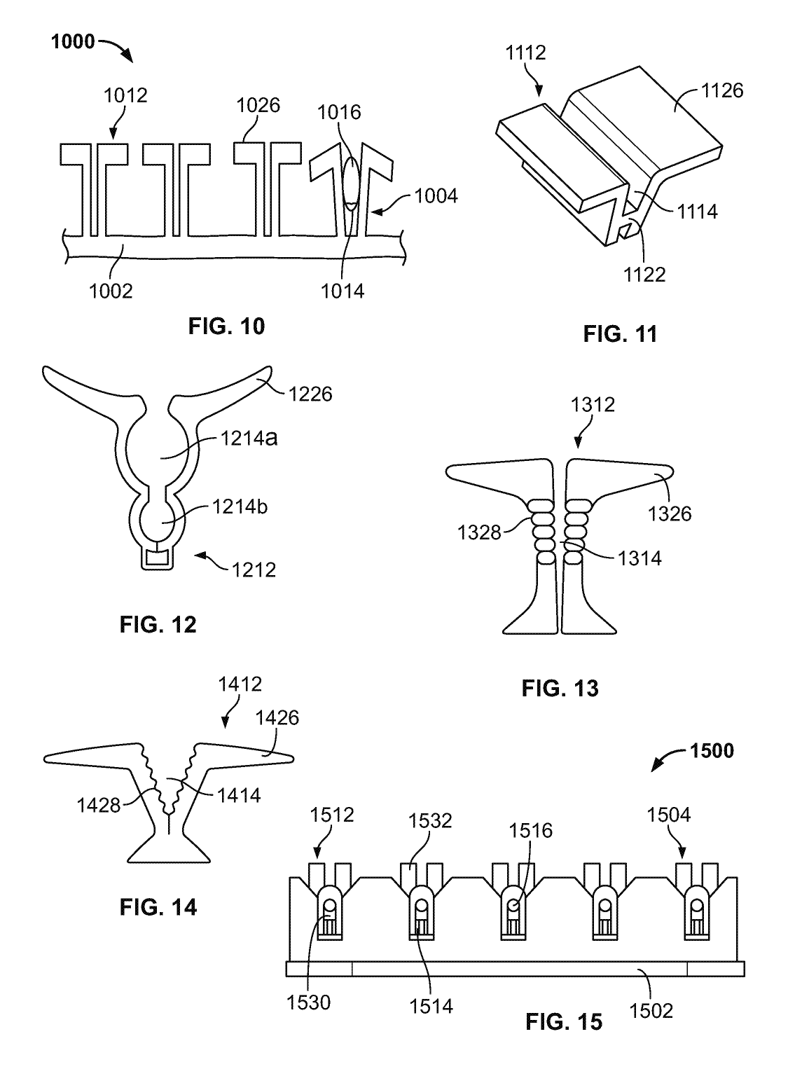

FIGS. 10-14 show perspective views of different retaining features of wall members with “gull wings.” These retaining features may be formed by shaping the structure of the wall member. These gull winged wall members may be viewed as a variation of the winged wall members in FIG. 6 or one-wing wall members in FIG. 8.

FIG. 10 shows a side view of an exemplary medical instrument holding apparatus that includes a gull winged wall member with an aperture. An exemplary medical instrument holding apparatus 1000 includes a base member 1002 and wall section 1004 extending from the base member 1002. The wall section 1004 may include a plurality of wall members 1012 extending from the base member 1002. Each wall member 1012 includes an aperture 1014 that receives an instrument 1016. The upper portion of the wall members 1012 includes “gull wings” upon which a user may grip to provide a counter tension to depress instrument 1016 into or lift instrument 1016 out of the aperture 1014. As depicted, when an instrument is inserted into the aperture 1014, the wall member 1012 is flexed outwardly to provide a larger aperture to receive the instrument. Instruments with larger shafts will outwardly flex the wall member more than instruments with smaller shafts.

FIG. 11 shows a three-dimensional side view of an exemplary gull winged wall member and a fulcrum within the aperture. FIG. 11 may be viewed as a variation of FIG. 10 with the addition of the fulcrum in FIG. 11. An exemplary wall member 1112 includes spaced apart sidewalls flanked at the top by a pair of wings 1126 and connected at the bottom by a bottom wall to form an aperture 1114 between the sidewalls. The bottom wall may support a fulcrum 1122 or the fulcrum may form the bottom wall. The fulcrum within the aperture 1114 eases the release of an instrument from the aperture.

A user may apply counter tension against one of the pair of wings 1126 using their thumb while depressing an instrument into the aperture 1114 onto the fulcrum 1122 using another finger of the same hand. Conversely, the user may apply counter tension against one of the wings 1126 using their thumb while lifting the instrument out of the aperture 1114. The force applied by the user is magnified and transferred via fulcrum 1122, thus minimizing the force required to lift the instrument out of the aperture 1114.

FIG. 12 is a side view of an exemplary gull winged wall member with a plurality of apertures. FIG. 12 may be view as a variation of FIG. 8 with a pair of gull wings in FIG. 12. An exemplary wall member 1212 includes a pair of wings 1226 at the upper portion and a plurality of apertures 1214a, 1214b for retaining instruments of differently sized shafts as discussed in FIG. 8.

FIG. 13 is a side view of an exemplary gull winged wall member with ribbed side walls. A wall member 1312 includes side walls flanked at the top by a pair of wings 1326 and an aperture 1314. The side walls include ribbed portions 1328 for gripping instruments of differently sized shafts. The ribbed portions 1328 may provide ribbed surfaces on both the inner and outer surfaces of both sidewalls. This may increase the flexing ability of the sidewalls.

In some embodiments, the bottom portion of the sidewalls may be connected with each other to define the bottom boundary of aperture 1314. In other embodiments, the sidewalls are spaced apart from each other, such that the base member forms the bottom boundary of aperture 1314. The sidewalls may be flared to form an aperture of increasing width from the bottom to the top of the wall member. This flared configuration may allow accommodation of differently sized instruments. In some embodiments, only one of the side walls includes a ribbed portion.

FIG. 14 is a side view of an exemplary gull winged wall member with inner surfaces of the wing member's sidewalls ribbed. FIG. 14 may be viewed as a variation of FIG. 13. In FIG. 13, both the inner and outer surfaces of the wall member's sidewalls are ribbed, whereas only the inner surfaces of the wall member's side walls are ribbed in FIG. 14. A wall member 1412 includes sidewalls flanked by a pair of wings 1426 at the top connected by a base portion at the bottom of the sidewalls near where the sidewalls meet. An aperture 1414 is formed by the sidewalls. The inner surfaces of the sidewalls that define the side boundaries of the aperture 1414 include a ribbed portion 1428. The ribbed inner surfaces may be integrally formed as part of the wall member. In some embodiments, only one of the side walls includes a ribbed portion.

FIGS. 15-19B show different mechanical retaining features that enhance apertures and their ability to retain and release medical instruments. FIGS. 15-18B show perspective view of spring operated engagement and release of medical instruments in the apertures of wall members.

FIG. 15 is a side view of an exemplary medical instrument holding apparatus that uses magnetic tubes and stops. A medical instrument holding apparatus 1500 includes a base member 1502 and wall section 1504 extending from the base member 1502. The wall section 1504 may include a plurality of wall members 1512 extending from the base member 1502. The wall members include a plurality of apertures 1514 as defined by its sidewalls. A stop 1530 is positioned at the bottom of each aperture. The stop 1530 may be spring loaded. Magnetic tubes 1532 are positioned along the periphery of each aperture to retain the medical instrument within the aperture.

A user may depress the medical instrument into the aperture 1514 by a downward force. The application of the downward force may be aided by a user applying counter tension on the top surface of the magnetic tubes while using another finger on the same hand to depress the medical instrument into the aperture 1514. The magnetic tubes 1532 may attract the medical instrument and secure it into the aperture 1514. Through a second application of a downward force to the instrument positioned on the spring loaded stop, the medical instrument 1516 may be ejected out the aperture. In some embodiments, the medical instrument holding apparatus may include a button to release the instrument.

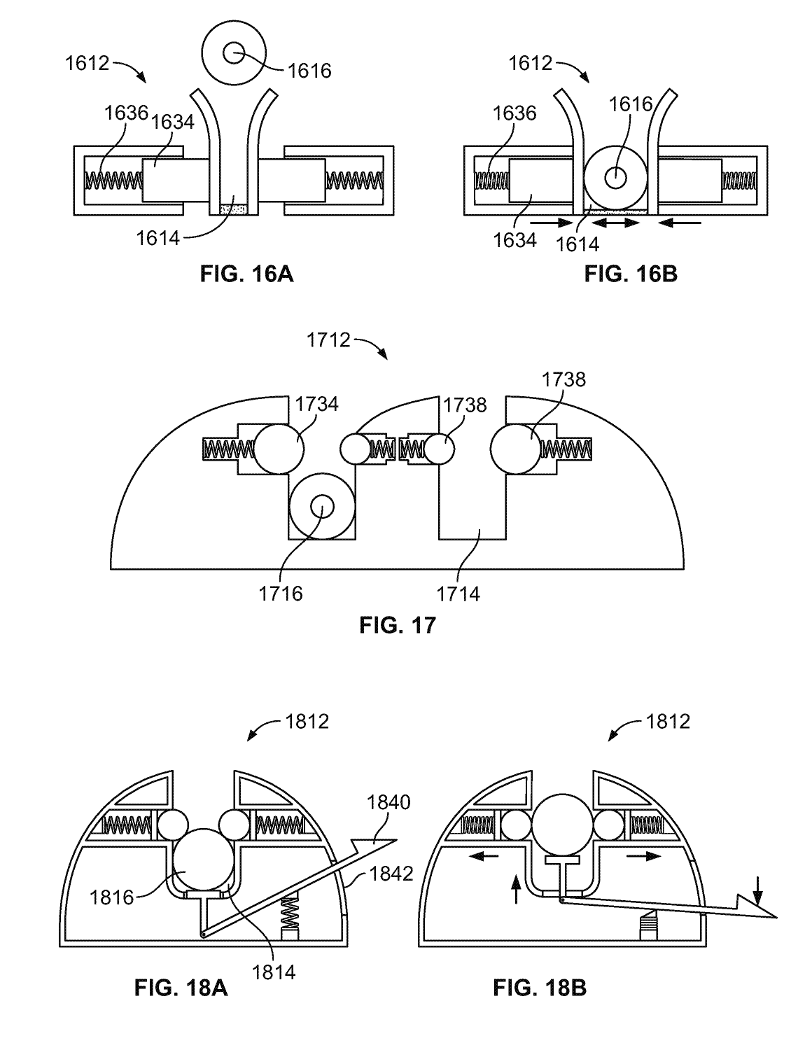

FIGS. 16A and 16B are perspective views of a wall member of a “sliding grip” arrangement. FIG. 16A is a perspective view of the wall member before receiving the medical instrument or after releasing the medical instrument. FIG. 16B is a perspective view of the wall member when engaging a medical instrument.

An exemplary wall member 1612 includes sidewalls that define an aperture 1614. The bottom of the aperture may include a bridge that connects the sidewalls. Each sidewall is abutted by a cam 1634, and each cam 1634 is connected to a spring 1636. The spring biased cams 1634 engages or cams the sidewalls to effect a linear bilateral displacement of the sidewalls. The springs 1636 may compress when an instrument 1616 is inserted into the aperture 1614 to securely grip the instrument 1616 (FIG. 16B) and return to its uncompressed state when the instrument 1616 is not in the aperture 1614 (FIG. 16A).

The upper portion of the sidewalls includes a pair of wings upon which a user's thumb may apply a counter tension against to insert a medical instrument 1616 into or lift the medical instrument 1616 out of the aperture 1614 using another finger on the same hand as the thumb.

In some embodiments, only unilateral displacement may be effected by the sidewalls. For example, only one of the sidewalls may be abutted with a spring biased cam so that the other sidewall remains fixed. In some embodiments, the inner surfaces of the sidewalls include foam grips to more firmly secure the medical instrument in the aperture. In certain embodiments, the sidewalls may not include the pair of wings. In some embodiments, the wall member may include a button to separate the sidewalls to allow insertion of the medical instrument into the aperture by compressing the springs. The button may decompress the springs to eject the medical instrument out of the aperture. In some embodiments, a lever may be positioned at the bottom of the aperture upon which the medical instrument rests. A downward application of force to the lever may provide a “scoop release” of the instrument out of the aperture. In some embodiments, the bottom boundary of the aperture is defined by the base member upon which the wall member rests.

FIGS. 17, 18A, and 18B are perspective view of a wall member of a “spring ball” arrangement. FIG. 17 is a side view of a wall member of a “spring ball” arrangement. FIGS. 18A and 18B are side views of a lever operated “spring ball” wall member. FIG. 18A is a side view of a lever operated “spring ball” wall member when the instrument is engaged. FIG. 18B is a side view of the lever operated “spring ball” wall member of FIG. 18A when the instrument is released.

As depicted in FIG. 17, an exemplary “spring ball” wall member 1712 includes sidewalls that define an aperture 1714. The upper portion of the sidewalls includes openings 1738 through which spring balls 1734 may engage with or release the instrument 1716. When the instrument is engaged in the wall member, the spring ball may protrude from the openings in the sidewalls above the instrument to prevent the instrument from being inadvertently lifted out the aperture. As discussed above, a user may apply counter tension against the top surfaces of the wall member to depress the medical instrument into and lift the medical instrument out of the aperture.

As depicted in FIGS. 18A and 18B, an exemplary lever operated “spring ball” wall member 1812 includes a lever 1840 forming the bottom boundary of an aperture 1814. When inserted into the aperture 1814, the aperture rests upon the fulcrum of the lever 1840. The lever 1840 extends through a side opening 1842 of an outer surface of the wall member. To lift the medical instrument 1816 out of the aperture, a user can apply a downward force to the lever so the medical instrument exerts a force against the spring balls to pass through them. The lever 1840 may be spring actuated to provide the momentum to release the medical instrument.

FIGS. 19A and 19B are perspective views of wall members using a “paddle mechanism” to retain medical instruments. An exemplary wall member 1912 includes an aperture 1914 between opposing sidewalls connected to a top end of a vertical shaft 1942. The sidewalls are connected with enclosure members 1948 that reduces the chances of a medical instrument 1916 from being inadvertently lifted out of the aperture 1914. The upper portion of the shaft 1942 has an upward-opening receptacle that receives the medical instrument 1916 and that supports the shaft of the medical instrument 1916. In the depicted example, the receptacle forms a curvature designed to conform to a cylindrical shaft of the instrument 1916 when inserted in the aperture 1914. The lower portion of the shaft 1942 has ratchet mechanism including a series of latching teeth 1946 that engage with a latch 1940. A pair of springs or elastic bands 1952 connects between the shaft 1942 and the enclosure members 1948. In operation, the shaft 1942 may be depressed downward when the instrument 1916 is loaded into the aperture 1916, thereby compressing a spring 1950 coupled to a bottom end of the shaft 1942. The latch 1940 may engage latching teeth 1946, for example, while the spring 1950 is under compression.

When an instrument is depressed into the holder 1944 of the shaft 1942, the shaft 1942 is moved downward. This causes the pair of springs or rubber bands 1952 to close the enclosure members 1948 across the instrument. A user may depress the lever 1940 to open the enclosure members 1948 so the user may access the medical instrument 1916. Depressing the lever 1940 may disengage the latch 1940 from the latching teeth 1946, and automatically eject the instrument 1916 out of the aperture 1914.

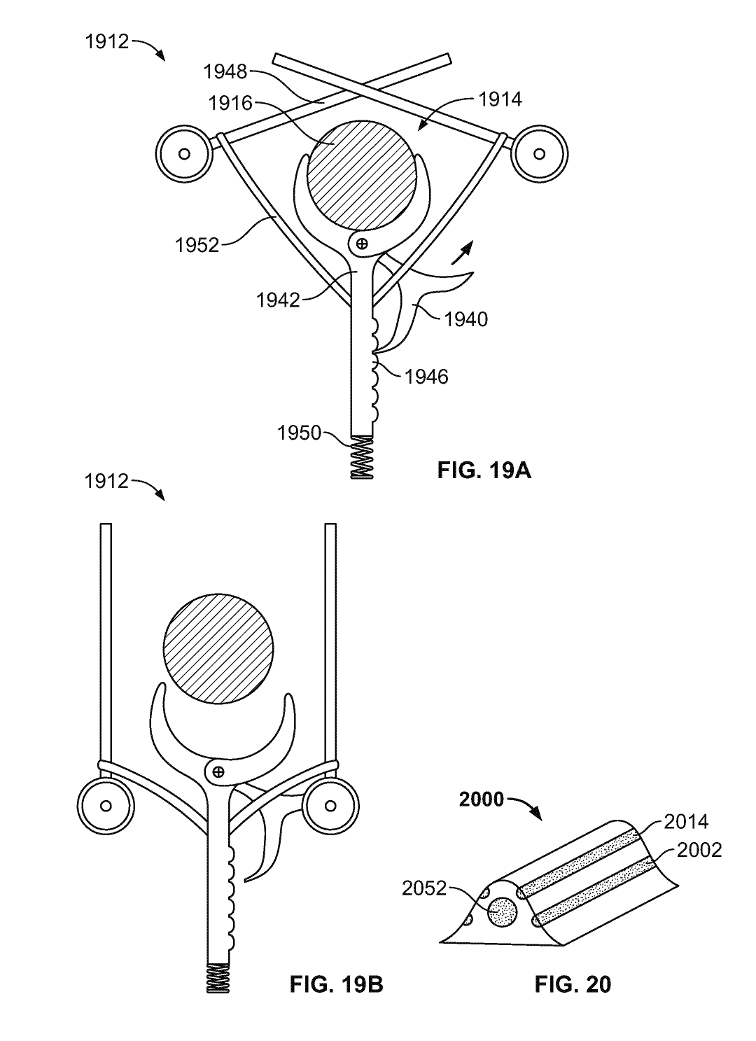

FIG. 20 is a three-dimensional view of an exemplary medical instrument holding apparatus with a parabolic cross section. An exemplary medical instrument holding apparatus 2000 includes a body 2002 with a parabolic cross section. The top surface of the body 2002 is defined by the curvature of a parabola with the vertex of the parabola positioned at the apex of the body 2002. A plurality of surface channels 2014 may be positioned along the upper surfaces of the body 2002 and extend longitudinally along the body 2002 to receive medical instruments. Tabs may be formed around and along the length of the channel that deflect to allow the shaft of the instrument to snap into the channel, giving a tactile, and potentially, audible response. An inner channel 2052 may extend through the center of the body for holding instruments of larger shaft diameters.

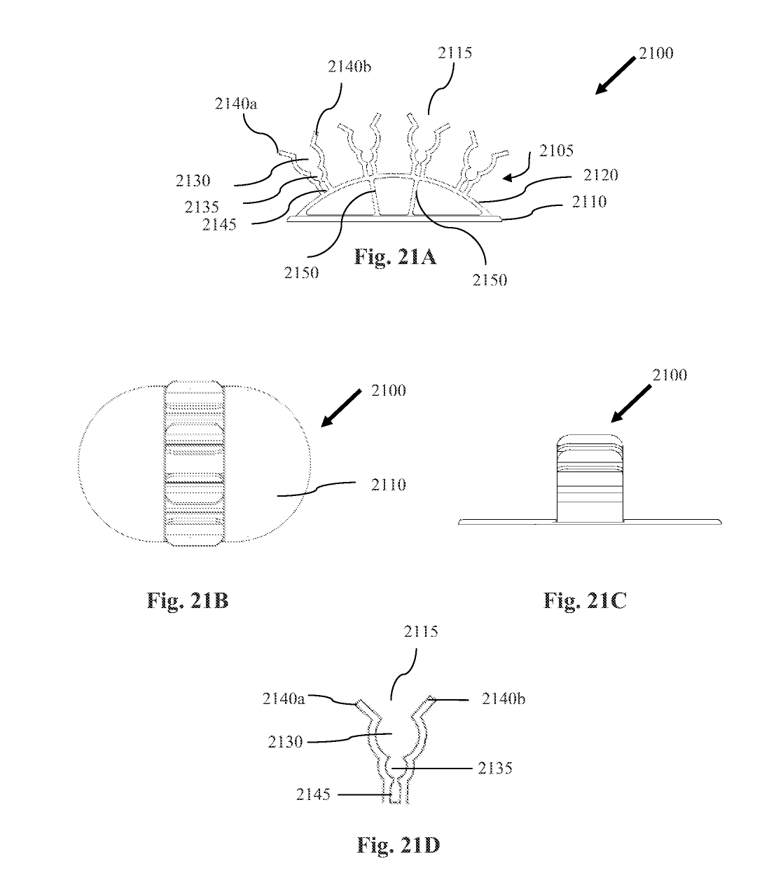

FIGS. 21A-21D show perspective views of an exemplary medical instrument holding apparatus, including a cross-section view along the length of a retention module in FIG. 21A, top view in FIG. 21B, and a side view in FIG. 21C. FIG. 21D shows a cross section view of an exemplary retention module.

An exemplary medical instrument holding apparatus 2100 includes an instrument support assembly (ISA) 2105 disposed on a base 2110. The ISA 2105 may be integrally formed (e.g., from a mold) with the base 2110. In some other examples, the ISA 2105 may be adhered to the base 2110 by a suitable fastening interface such as, for example, an adhesive material, alone or in combination with one or more clips, snaps, or hook and loop fasteners. In the depicted example as shown in FIG. 21B, the base 2110 is substantially planar member in an elliptical shape.

As depicted in the figure, the ISA 2105 includes a plurality of retention modules 2115 arranged in a radial geometry around an exterior surface of a non-planar platform 2120. Each of the retention modules 2115 is configured to hold one or more instruments. In various examples, each retention module 2115 may securely hold more than one instrument. In some implementations, each held instrument may have a substantially different diameter at the point on the shaft retained by the retention module 2115. In some examples, each of the retention modules 2115 may be sized and configured to retain instrument shafts of different shapes with respect to one or more of the other retention modules 2115. When placed into a retention module 2115, an instrument's shaft may be substantially parallel to the longitudinal axis of the base 2110. The use of a single row of retention modules 2115 may, in some applications, provide for rapid and secure engagement and disengagement of instruments using, for example, one hand, with tactile and/or audible feedback to provide a non-visual indication of secure engagement, for example, of an instrument in the retention module 2115.

In the depicted example as shown in FIG. 21D, a retention module 2115 includes a first cavity 2130, a second cavity 2135, finger tabs 2140a, b, and a foot 2145. The first cavity 2130 and the second cavity 2135 are of different sizes so they may simultaneously accommodate instruments with shafts having different diameters. The retention module 2115 is flanked by finger tabs 2140a, b at its upper portion. The finger tabs 2140a, b may include beveled or rounded edges, for example, to prevent catching onto or scratching people or other things. For example, sharp edges may catch onto clothing or scratch fingers. The retention module 2115 may provide tactile and/or audible feedback when instruments are properly engaged into or disengaged from either the first cavity 2130 or the second cavity 2135. To remove the instrument, a user may apply their thumb against the finger tab 2140a or 2140b while using their index finger for example to lift the instrument out of the retention module 2115. The retention module 2115 is connected to the platform 2120 by the foot 2145.

In the depicted example as shown in FIG. 21A, the platform 2120 is arc-shaped so that the retention modules 2115 are not coplanar no plane can intersect each of the retention modules 2115 at its respective joint with the platform 2120). Stated in another way, the retention modules 2125 are supported by the platform at different attitudes with respect to the base 2110. In the depicted FIG. 21A, each of four respective tines that bisect each of the four respective retention modules 2115 intersect according to a cylindrical geometry; accordingly, such bisecting lines are non-parallel. In some other embodiments, a first group of the retention modules 2115 may be supported by the platform at a first altitude with respect to the base 2110, and a second group of the retention modules 2115 may be supported by the platform 2120 at a second altitude with respect to the base 2110, where the first and second altitudes are substantially different. In some examples, a line that bisects one of the retention modules in the first group may be substantially parallel to a tine that bisects one of the retention modules in the second group. Accordingly, improved densities may be achieved with adjacent retention modules being supported in two or more support planes located at different altitudes with respect to the base 2110.

In some applications, the retention modules 2115 that are non-coplanar with each other may advantageously provide retention modules at differing heights or angles relative to the longitudinal axis of base 2110. With retention modules 2115 being non-coplanar with each other, the spacing between retention modules 2115 may be reduced without interference between instruments being retained in adjacent ones of the retention modules 2115. Thus, non-coplanarity of the retention module 2115 may advantageously yield higher instrument storage density with the ability of the medical instrument holding apparatus 2100 to retain more surgical instruments in a smaller space or volume. Such a high density instrument retention may, in some implementations, yield a more compact device with a smaller footprint that is easier to position, install, manage, or dispose of. In some examples, a reduced size may in turn reduce a cost and/or weight of the apparatus 2100, while increasing the convenience and/or flexibility in locating instruments in the surgical environment.

In the depicted example, the platform 2120 includes ribs 2150 to provide strength and integrity to support the weight of the retention modules above. In some examples, the platform 2120 may be a solid surface.

In some examples, the base 2110 may be made of a non-slip material to preventing slipping of the apparatus against an attachment surface, such as the patient, covers over a patient, or surgical table, tray, or cart. In some examples, the bottom of the base 2110 may include fasteners, such as, for example, suction cups, bolts, adhesive, snaps, and/or hooks in a hook and loop fastener system, or a combination of these.

In some examples, the base 2110 may have a shape other than elliptical, such as, for example, rectangular or triangular. In some embodiments, the platform 2120 may be a shape other than arc-shaped, such as, for example, step configuration, pyramidal, and other shapes to create non-coplanar retention modules.

In some embodiments, the retention modules 2115 of the ISA 2105 may define different sized cavities (e.g., the cavities 2130, 2135) and different orientation of cavities within a single retention module 2115 and/or among a group of the retention modules 2115 in the ISA 2105. In some examples, there may be one, two, three, five, six, seven, eight, nine, ten, or more retention modules 2115 in the ISA 2105. In some examples, the smaller cavity may be distal to the larger cavity in a retention module with respect to the platform 2120. In some examples, the cavities in the retention module may be of the same size. In some examples, the retention module 2115 may have one, three, four, five, six, seven, eight, nine, ten, or more cavities. In some examples, one or more of the cavities in the retention module 2115 may have a geometry that other than substantially circular. For example, one or more cavities in the retention module 2115 may be formed into a substantially triangular, square, rhombus, pentagon, hexagon, octagon, or similar N-sided polygon or irregular shape (e.g., oval, double barreled) to provide an effective securement and ready release for instrument shafts of various sizes and form factors.

Although various embodiments have been described with reference to the figures other implementations are contemplated. For example, the construction of the holder apparatus may be of a pliable material, such as foam. In some examples, an extruded plastic core may be coated with a pliable material that can deform and return to their original shape. In some embodiments, surfaces for gripping the medical instrument may be selectively coated to provide a surface with a substantially increased coefficient of friction to more securely grip the instrument. By way of example, and not limitation, channel surfaces such that are described herein may be treated with a rubber coating.

The medical instruments holding apparatus is not limited to holding tools for performing laparoscopic surgery. For example, the apparatus may be used in performing thoracoscopic and endoluminal surgeries. In various embodiments, the surgical tools being secured and released may be generally symmetrical.

In some embodiments, a user receives confirmation that a medical instrument is secured within a medical instrument holding apparatus. This confirmation may be in the form of audible, tactile, and/or visual feedback. For example, audible feedback may include a chirp, music, tone, or snap sound. Tactile feedback may include pressure sensitive or snap sensation. Visual feedback may include light or other visual indicator that indicates the medical instrument has been secured or released.

In some embodiments, the medical instrument holding apparatus may be disposable. In other embodiments, the medical instrument holding apparatus may be formed from autoclavable materials.

In some embodiments, the medical instrument holding apparatus may include a base member with at least one watt section that has at least one wall member. Each wall member may be permanently or detachably attached as units in a wall section. Each wall section may be permanently or detachably attached from the base member. In other embodiments, the medical instrument holding apparatus includes separate wall member units that can be individually and separately attached to various locations on the drape or nearby surgical table. In further embodiments, individual wall member units or base member with at least one wall section that has at least one wall member or the individual wall section may be integral with drape.

A number of implementations have been described. Nevertheless, it will be understood that various modification may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A medical instrument holding apparatus comprising:
a base member including a top surface and a bottom surface, the top surface having two opposite sides and two opposite ends and the bottom surface have an adhesive protected by a sheet;
a wall section formed along each of the two opposite ends, each wall section comprising a plurality of wall members having sidewalls that define a plurality of spaced apart apertures, each of the apertures of each wall section being laterally aligned with each of the apertures of the other wall section to retain a medical instrument at two positions;
a tab positioned at the upper surface of each wall section end; and,
a retention mechanism within each aperture that is configured to provide feedback when a medical instrument is engaged with or released from an aperture with downward force,
wherein each of the tabs is configured for receiving downward force exerted by a user's finger as counter tension while the user depresses a medical instrument into an aperture or lifts the medical instrument out of the aperture with another finger on the same hand as the thumb.

2. The apparatus of claim 1, wherein the upper portion of each of e wall members includes a pair of tabs.

3. The apparatus of claim 1, wherein the retention mechanism comprises a pair of internal ribs formed as part of the sidewalls of the walls members.

4. The apparatus of claim 1, wherein the retention mechanism comprises magnetic tubes within the aperture for attracting the medical instrument.

5. The apparatus of claim 1, wherein the retention mechanism comprises a spring ball mechanism that grips the medical instrument.

6. The apparatus of claim 1, wherein each of the wall members comprises a plurality of differently sized apertures for holding instruments of differently sized shafts.

7. The apparatus of claim 1, wherein the base member comprises resting pads on each side of the wall section.

8. The apparatus of claim 1, wherein the base member comprises a plurality of fulcrums on each side of the wall section.

9. The apparatus of claim 1, wherein the lower portion of the wall member comprises a fulcrum.

10. The apparatus of claim 1, wherein the inner surfaces of the wall members are ribbed.

11. The apparatus of claim 1, wherein the wall members comprises of cylindrical projections.

12. The apparatus of claim 1, wherein the walls sections are staggered.

13. The apparatus of claim 1, wherein the wall members comprises a lever for releasing the medical instrument from the aperture.

14. A method comprising:
providing a base member including a top surface and a bottom surface, the top surface having two opposite sides and two opposite ends and the bottom surface have an adhesive protected by a sheet;
providing a wall section formed along each of the two opposite ends, each wall section comprising a plurality of wall members having sidewalls that define a plurality of spaced apart apertures, each of the apertures of each wall section being laterally aligned with each of the apertures of the other wall section to retain a medical instrument at two positions;
providing a tab positioned at the upper surface of each wall section end; and,
providing a retention mechanism within each aperture that is configured to provide feedback when a medical instrument is engaged with or released from an aperture with downward force,
wherein the tab receives the downward force exerted by a user's thumb as counter tension white the user depresses a medical instrument into an aperture or lifts the medical instrument out of the aperture with another finger on the same hand as the thumb.

15. A method comprising:
positioning a medical instrument holding apparatus on a sterile field, the medical instrument holding apparatus comprising:
a base member including a top surface and a bottom surface, the top surface having two opposite sides and two opposite ends and the bottom surface have an adhesive protected by a sheet;
a wall section formed along each of the two opposite ends, each wall section comprising a plurality of wall members having sidewalls that define a plurality of spaced apart apertures, each of the apertures of each wall section being laterally aligned with each of the apertures of the other wall section to retain a medical instrument at two positions;
a tab positioned at the upper surface of each wall section end; and,
a retention mechanism within each aperture that is configured to provide feedback when a medical instrument is engaged with or released from an aperture with downward force,
positioning a medical instrument into an aperture of the medical instrument holding apparatus using one hand by applying counter tension on a tab using a thumb while depressing the medical instrument using another finger of the same hand; and,
receiving feedback that the medical instrument is secured into the aperture.
